(19) United States
(12) Patent Application Publication
(10) Pub. No.: US 2008/0051319 A1
(43) Pub. Date: Feb. 28, 2008

(54) Inhibiting JNK Signaling Promotes CNS Axon Regeneration

(75) Inventors: Zhlgang He, Boston, MA (US); Glenn Yiu, Boston, MA (US)

Correspondence Address: RICHARD ARON OSMAN, 4070 CALLE ISABELLA, SAN CLEMENTE, CA, 92672, US

(73) Assignee: Children's Medical Center Corporation, Boston, MA (US)

(21) Appl. No.: 11/842,542

(22) Filed: Aug. 21, 2007

Related U.S. Application Data
(60) Provisional application No. 60/839,595, filed on Aug. 22, 2006.

(51) Int. Cl. A61K 38/02; A61K 31/403; A61K 31/416; C12Q 1/48; A61P 25/00; A61K 31/506

(52) U.S. Cl. 514/2; 435/15; 514/275; 514/406; 514/411; 514/789

(57) ABSTRACT

Regeneration of a lesioned CNS axon of mature neuron, determined to be subject to regeneration inhibition by endogenous cJun-N-terminal kinase (JNK), is promoted by contacting the neuron with an exogenous JNK inhibitor at a concentration sufficient to partially inhibit the JNK, and thereby promote a resultant regeneration of the axon.

This Application is a continuation of U.S. Ser. No. 60/839,595 filed Aug. 22, 2006.

This work was supported by NIH/NINDS grant no. 1R01NS051788. The U.S. government may have rights in any patent issuing on this application.

### BACKGROUND OF THE INVENTION

The field of the invention is inhibition of cJun-N-terminal kinase (JNK) activity to promote regeneration of a lesioned CNS axon of a mature neuron.

Adult CNS injury often results in the exposure of severed axons to a variety of myelin-derived inhibitory molecules which can severely limit axon repair. Many of these molecules including Nogo-A, MAG, and OMgp [1-3] can bind to the neuronal receptor NgR [4-7], and trigger intracellular signals in neurons that ultimately result in their failure to regenerate. The best characterized pathway so far involves the small GTPase RhoA and its effector, RhoA-associated kinase (ROCK) [8-12]. Small GTPases of the Rho family such as RhoA, Rac1, and Cdc42 are known regulators of the actin cytoskeleton [13]. Recent work has shown that RhoA activation can signal through LIM-kinase and Slingshot (SSH) phosphatase to regulate the actin depolymerization factor cofilin [14]. Together, these reports support a model where myelin-associated molecules converge on the NgR complex to activate RhoA, stabilizing the growth cone cytoskeleton of damaged axons at the lesion site, and preventing nerve fiber regeneration. However, it remains unclear how the NgR complex triggers RhoA activation, or whether myelin inhibitors have effects that act beyond the actin cytoskeleton.

Since NgR is a GPI-linked molecule and lacks an intracellular domain, it must rely on transmembrane co-receptors to transduce the inhibitory signals. The identification of two TNFR family members, p75 [2, 15-17] and TROY [18, 19], as functionally homologous co-receptors for NgR suggests that intracellular domains shared by both molecules may be involved in triggering downstream signals like RhoA. An early report suggested that p75 can interact with a Rho guanine dissociation inhibitor (Rho-GDI) [20], displacing it from its inhibition of RhoA. However, it is not known whether a guanine nucleotide exchange factor (GEF) may also be required to activate RhoA after its dissociation from Rho-GDI. It is also unclear whether TROY can signal to RhoA in a similar manner.

Based on their cytoplasmic sequences and signaling properties, TNF receptors can be classified into two major groups. Some members have a cytoplasmic death domain which can trigger the caspase signaling cascade. Other members such as p75 and TROY contain intracellular motifs that interact with TNF-receptor associated factors (TRAFs). Receptor recruitment of these adaptor molecules leads to the activation of signaling mediators such as JNK and NF-κB [21-23].

JNK activation has been implicated in a variety of signaling networks, and is associated with the phosphorylation of microtubule-associated proteins (MAPs) such as MAP2 [26], Tau [27], and doublecortin [28], as well as stress-related transcription factors like cJun [29]. JNK is enriched in neuronal axons and can associate with motor proteins [30]. A number of reports have shown that nerve fiber transection triggers a characteristic axonal response that leads to dramatic changes in the transcription program of the injured neuron [31-33]. However, it is not known whether these effects result from the interruption of constitutive retrograde signals in the severed axons, or from positive electrical or molecular injury signals arising from the lesion site. It is also unclear how these signals may be converted into a transcriptional response. JNKs have been implicated in the initiation of this axonal response because they are rapidly activated following nerve injury and may be transported along microtubules through their association with motor proteins [30]. At the same time, immediate-early transcription factors of the AP-1 family including cJun are also highly induced in response to neuronal injury, and in part mediate the transcriptional response [34]. Current models postulate that cell stress and inflammatory signals trigger the activation and retrograde transport of JNK along axons to phosphorylate cJun at the cell body [35]. JNK and cJun have been linked to axotomy-induced cell death and axonal regeneration [36]. We have discovered a novel role for JNK activation in mediating NgR-dependent signals for mediating NgR-dependent signals for myelin inhibition.

US Pat Publ No. 20060122179 to Zeldis et al. proposes treating or preventing a CNS injury, including axonal injury, by administering a therapeutically or prophylactically effective amount of a JNK inhibitor to a patient. In their disclosed animal (rat) experiments Zeldis et al used 10 mg/kg dosages, which they translate to final plasma and brain concentrations of 7 and 65 uM, respectively. Zeldis elsewhere recites enormous dosage ranges of “about 1 mg to about 10,000 mg per day” (para 0248).

JNK inhibition has been reported to reduce neuronal apoptotic death in several neurodegenerative diseases and ischemic brain damage (see e.g. Yang et al, Proc. Natl. Acad. Sci. U. S. A. (1997) 94:3004-3009; Yang et al., Nature (1997) 389:865-870; Okuno et al, J. Neurosci. (2004) 24:7879-7887; Saporito et al, J Pharmacol Exp Ther (1999) 288:421-427; and Gao et al, J. Cereb. Blood Flow Metab. (2005) 25:694-712).

Yin et al (Neurobiol Dis. (2005) 20:881-9) administered JNK inhibitors 12 hours prior to inducing spinal cord injury in rats. Their results suggested that JNK activation contributes to trauma-induced DP5 expression and subsequent apoptosis in spinal cord injury. Yin suggests that the JNK signaling pathway may be a potential target for therapeutic interventions for spinal cord injury, and proposes further studying the effect of JNK inhibition after spinal cord injury. Yin's rats received 15 mg/kg dosages of SP600125.

Obata et al. (J. Neurosci., 24 Nov. 2004(45):10211-22) report that sustained intrathecal administration of SP600125 (2.5 ug/ul (11 mM)×hr) inhibited mechanical hypersensitivity at 3 and 7 d after spinal nerve ligation surgery, while SP600125-treatment did not affect thermal hyperalgesia.

Lindwall et al. [35] reported that inhibition of c-Jun phosphorylation with the selective JNK inhibitors SP600125 and (D)-JNKI1 dramatically reduced axonal outgrowth in explanted or dissociated ganglia sensory neurons.

### SUMMARY OF THE INVENTION

One aspect of the invention is a method of promoting regeneration of a lesioned CNS axon of a mature neuron determined to be subject to regeneration inhibition by endogenous cJun-N-terminal kinase (JNK). The method comprises the steps of: (a) contacting the neuron with an exogenous JNK signaling pathway inhibitor at a concentration sufficient to only partially inhibit the JNK signaling, and thereby promote a resultant regeneration of the axon; and (b) detecting the resultant regeneration of the axon. In one embodiment the inhibitor is SP600125 at a nanomolar concentration.

In various embodiments, the lesion results from a traumatic injury, an acute spinal cord injury, or CNS degeneration.

In a specific embodiment, the lesioned axon is in the spinal cord of a patient, and the inhibitor is intrathecally administered to the patient.

In various embodiments, the axon is a CNS axon of a sensory neuron, or a CNS axon of a cerebellar granule neuron.

The detecting step may be effected by an indirect or direct assay of axon regeneration.

### DETAILED DESCRIPTION OF SPECIFIC EMBODIMENTS OF THE INVENTION

We have found that myelin-associated inhibitors can trigger the activation of the cJun-N-terminal kinase (JNK), a common downstream effector of tumor necrosis factor receptor (TNFR) members, in a Nogo-66 receptor-(NgR) dependent manner. We show that partial, but not complete, pharmacological blockade of JNK signaling neutralizes neurite outgrowth inhibition and RhoA activation by myelin components. Our invention provides methods and compositions for promoting regeneration of a lesioned CNS axon of a mature neuron determined to be subject to regeneration inhibition by endogenous cJun-N-terminal kinase (JNK). The method comprises the steps of: contacting the neuron with an exogenous JNK signaling pathway inhibitor at a concentration sufficient to only partially inhibit the JNK signaling, and thereby promote a resultant regeneration of the axon; and detecting the resultant regeneration of the axon.

The lesioned CNS axon is subject to regeneration inhibition by JNK, which may be detected directly, indirectly, or inferred. For example, activated JNK in a neuron can be detected by antibody specific for phosphorylated JNK. Alternatively, the presence of activated JNK in the neuron may be inferred where the lesioned axon is in contact with injured myelin. In one example, the lesioned axon is a CNS axon of a dorsal root ganglion (DRG) sensory neuron. In another example, the lesioned axon is a CNS axon of a cerebellar granule neuron. The mature (i.e. terminally-differentiated, non-embryonic) neuron may be in vitro or in situ in a patient. In specific embodiments, the patient is a mammal (e.g. human, companion animal, livestock animal, rodent or primate animal model for neurodegeneration or CNS injury, etc.).

The lesion can result from traumatic injury, optic nerve injury or disorder, brain injury, stroke, chronic neurodegeneration such as caused by neurotoxicity or a neurological disease or disorder (e.g. Huntington's disease, Parkinson's disease, Alzheimer's disease, multiple system atrophy (MSA), etc.).

In one embodiment, the inhibitor is used to treat an ocular injury or disorder (e.g. toxic amblyopia, optic atrophy, higher visual pathway lesions, disorders of ocular motility, third cranial nerve palsies, fourth cranial nerve palsies, sixth cranial nerve palsies, internuclear ophthalmoplegia, gaze palsies, eye damage from free radicals, etc.), or an optic neuropathy (e.g. ischemic optic neuropathies, toxic optic neuropathies, ocular ischemic syndrome, optic nerve inflammation, infection of the optic nerve, optic neuritis, optic neuropathy, papilledema, papillitis, retrobulbar neuritis, commotio retinae, glaucoma, macular degeneration, retinitis pigmentosa, retinal detachment, retinal tears or holes, diabetic retinopathy, iatrogenic retinopathy, optic nerve drusen, etc.).

In a particular embodiment, the lesion results from acute or traumatic injury such as caused by contusion, laceration, acute spinal cord injury, etc. In specific embodiments, the lesioned CNS axon is in CNS white matter, particularly white matter that has been subjected to traumatic injury. In certain embodiments, the contacting step is initiated within 96, 72, 48, 24, or 12 hours of formation of the lesion.

Our methods allow treatment even after neuronal apoptosis; hence, treatment with a JNK inhibitor may be initiated or continued subsequent to cessation of apoptosis in indications that are associated with neuronal apoptosis. In various indications, the contacting step is initiated, and/or treatment is continued, more than 5, 7, 14, 30, or 60 days after formation of the lesion.

The inhibitor can be administered to the injured neuron in combination with, or prior or subsequent to, other treatments such as the use of anti-inflammatory or anti-scarring agents, growth or trophic factors, etc. In a specific embodiment, the lesion results from acute spinal cord injury and the method additionally comprises contacting the neuron with methylprednisolone sufficient to reduce inflammation of the spinal cord. In various other embodiments, the inhibitor is administered in combination with trophic and/or growth factors such as NT-3 (Piantino et al, Exp Neurol. (2006) June 7; [Epub ahead of print]), inosine (Chen et al, Proc Natl Acad Sci USA. (2002) 99:9031-6; U.S. Pat. No. 6,551,612 to Benowitz; U.S. Pat. No. 6,440,455 to Benowitz; and US Pat Publ 20050277614 to Benowitz), oncomodulin (Yin et al, Nat Neurosci. (2006) 9:843-52.; US Pat Publ 20050054558 to Benowitz; US Pat Publ 20050059594 to Benowitz; and U.S. Pat. No. 6,855,690 to Benowitz), etc.

JNK inhibitors are well-known in the art (see e.g. U.S. Pat. No. 6,987,184 to Sakata et al; U.S. Pat. No. 7,084,159 to Cao et al; US Pat Publ No. 20050148624 to Itoh et al; US Pat Publ No. 20060122179 to Zeldis et al. and Kuan and Burke, Curr Drug Targets CNS Neurol Disord (2005) 4:63-7). Exemplary JNK pathway inhibitors include CEP-1347 (Maroney et al, J Neurosci. (1998) 18:104-11), SP600125 (Bennett et al, Proc Natl Acad Sci USA. (2001) 98:13681-6), AS601245 (Carboni et al, J Pharmacol Exp Ther. (2004) 310:25-32), DJNK1 (Manning and Davis, Nat Rev Drug Discov. (2003) 2:554-65), AS-602801 (Halazy, ARKIVOC (2006) vii:496-508), XG-102 (Borsello et al, Nat Med. (2003) 9:1180-6.), AM-111 (Coleman et al, Hear Res. (2006) July 11; [Epub ahead of print]), CC-401 (Uehara et al, J Hepatol. (2005) 42:850-9), and CEP-11004 (Ganguly et al, J Neurochem. (2004) 88:469-80). In one embodiment, the inhibitor is siRNA targeted to a JNK pathway member (e.g. JNK1, JNK2, and JNK3).

The inhibitor is preferably used at a concentration that permits some basal level of JNK activity, i.e. the inhibitor only partially inhibits the JNK signaling and does not result in complete JNK inhibition. Concentrations of a specific inhibitor that achieve partial inhibition of JNK signaling are readily determined using assays such as a neurite outgrowth assay (e.g. see Example 1). Many inhibitors provide a bell-shaped titration curve beginning with a dose-dependent concentration range in which increasing inhibitor concentration results in increased neutralization of myelin-associated outgrowth inhibition and hence, increased axon regeneration, followed by an inversion wherein increasing inhibitor concentration results in decreasing degrees of axon regeneration until there is complete JNK inhibition and no detectable resultant axon regeneration. Preferred inhibitors achieve partial inhibition of JNK signaling at nanomolar or micromolar concentrations. In a specific embodiment, the JNK inhibitor is SP600125 that contacts the neuron at nanomolar concentrations. Preferably, the inhibitor does not inhibit signaling of p38 MAPK.

The inhibitor is contacted with the neuron using a suitable drug delivery method and treatment protocol sufficient to promote regeneration of the axon. For in vitro methods, the inhibitor is added to the culture medium, usually at nanomolar or micromolar concentrations. For in situ applications, the inhibitor can be administered orally, by intravenous (i.v.) bolus, by i.v. infusion, subcutaneously, intramuscularly, ocularly (intraocularly, periocularly, retrobulbarly, intravitreally, subconjunctivally, topically, by subtenon administration, etc.), intracranially, intraperitoneally, intraventricularly, intrathecally, by epidural, etc.

Depending on the intended route of delivery, the compositions may be administered in one or more dosage form(s) (e.g. liquid, ointment, solution, suspension, emulsion, tablet, capsule, caplet, lozenge, powder, granules, cachets, douche, suppository, cream, mist, eye drops, gel, inhalant, patch, implant, injectable, infusion, etc.). The dosage forms may include a variety of other ingredients, including binders, solvents, bulking agents, plasticizers etc.

In a specific embodiment, the inhibitor is contacted with the neuron using an implantable device that contains the inhibitor and that is specifically adapted for delivery to a CNS axon of neuron. Examples of devices include solid or semi-solid devices such as controlled release biodegradable matrices, fibers, pumps, stents, adsorbable gelatin (e.g. Gelfoam), etc. The device may be loaded with premeasured, discrete and contained amounts of the inhibitor sufficient to promote regeneration of the axon. In a particular embodiment, the device provides continuous contact of the neuron with the inhibitor at nanomolar or micromolar concentrations, preferably for at least 2, 5, or 10 days.

The subject methods typically comprise the further step of detecting a resultant regeneration of the axon. For in vitro applications, axonal regeneration may be detected by any routinely used method to assay axon regeneration such as a neurite outgrowth assay. For in situ applications, axonal regeneration can be detected directly using imaging methodologies such as MRI, or indirectly or inferentially, such as by neurological examination showing improvement in the targeted neural function. The detecting step may occur at any time point after initiation of the treatment, e.g. at least one day, one week, one month, three months, six months, etc. after initiation of treatment. In certain embodiments, the detecting step will comprise an initial neurological examination and a subsequent neurological examination conducted at least one day, week, or month after the initial exam. Improved neurological function at the subsequent exam compared to the initial exam indicates resultant axonal regeneration. The specific detection and/or examination methods used will usually be based on the prevailing standard of medical care for the particular type of axonal lesion being evaluated (i.e. trauma, neurodegeneration, etc.).

The invention also provides inhibitor-eluting or inhibitor-impregnated CNS implantable solid or semi-solid devices. Examples of CNS implantable devices include polymeric microspheres (e.g. see Benny et al., Clin Cancer Res. (2005) 11:768-76) or wafers (e.g. see Tan et al., J Pharm Sci. (2003) 4:773-89), biosynthetic implants used in tissue regeneration after spinal cord injury (reviewed by Novikova et al., Curr Opin Neurol. (2003) 6:711-5), biodegradable matrices (see e.g. Dumens et al., Neuroscience (2004) 125:591-604), biodegradable fibers (see e.g. U.S. Pat. No. 6,596,296), osmotic pumps, stents, adsorbable gelatins (see e.g. Doudet et al., Exp Neurol. (2004) 189:361-8), etc. Preferred devices are particularly tailored, adapted, designed or designated for CNS implantation. The implantable device may contain one or more additional agents used to promote or facilitate neural regeneration. For example, in one embodiment, an implantable device used for treatment of acute spinal cord injury contains the inhibitor and methylprednisolone or other anti-inflammatory agents. In another embodiment, the implantable device contains the inhibitor and a nerve growth factor, trophic factor, or hormone that promotes neural cell survival, growth, and/or differentiation, such as brain-derived neurotrophic factor (BDNF), ciliary neurotrophic factor (CNTF), nerve growth factor (NGF), inosine, oncomodulin, NT-3, etc.

### EXAMPLE 1

### Inhibition of JNK Blocks Outgrowth Inhibition by CNS Myelin

JNK is a member of the mitogen-activated protein kinase (MAP kinase) family which is abundantly expressed in axons and growth cones, and has been implicated in numerous roles including cell stress and cell proliferation. To determine whether this signaling component may be required for the inhibitory effects of CNS myelin, we tested the effects of a specific pharmacological inhibitor of JNK, SP600125 (IC₅₀=40 nM for JNK-1 and -2, and 90 nM for JNK-3 [24-25]), in an in vitro neurite outgrowth assay performed as described previously [6, 15]. Briefly, 4 week-old rat DRGs were dissected, dissociated and plated onto immobilized substrates (AP (100 ng/cm²) or AP-Nogo-66 (100 ng/cm²)). Cells were cultured for 24 h before fixation with 4% paraformaldehyde and staining with a neuronal-specific anti-β-tubulin III antibody (Tuj-1, Covance). The average lengths of the longest neurite in individual neurons were measured from at least 150 neurons per condition, from duplicate wells and from three independent experiments, and quantified as described previously [6, 15]. Immunohistochemistry was performed using antibodies against p75 (Chemicon).

We found that treatment with SP600125 significantly reduces the inhibitory effects of Nogo-66, the inhibitory extracellular domain of Nogo-A. The drug effect is dose-dependent at concentrations between 100 nM and 500 nm, and neutralizes outgrowth inhibition in both p75-positive (p75+) and p75-negative (p75−) neurons. Concentrations of SP600125 above 5 μM resulted in an abortion of neurite extension indicating that some JNK activation is required for basal axon elongation. This is consistent with previous studies which showed that JNK and cJun are actually required for neurite outgrowth [35]. It is also possible that the higher concentrations of SP600125 inhibited MKK3 and MKK6, which are activators of the p38 MAP kinase signaling pathway (Kuan and Burke, Curr Drug Targets CNS Neurol Disord (2005) 4:63-7); inhibition of p38 MAPK has been demonstrated to inhibit growth cone formation after axotomy (see Verma et al, J Neurosci. (2005) 25:331-42).

To determine whether JNK is activated in response to myelin inhibitors, serum-starved CGNs were treated with recombinant Nogo-66 at different dosages and time points, and the resulting lysates were directly used in Western blotting analysis and immunostained with anti-phospho-JNK and anti-JNK antibodies. Nogo-66 treatment triggered the phosphorylation of JNK in time- and dose-dependent manners. This activation is dependent on NgR, as lentivirus-mediated over-expression of a truncated dominant-negative NgR (DN-NgR), but not full-length NgR (FL-NgR) could efficiently block the phosphorylation of JNK. The JNK response to myelin inhibitors is only reduced in neurons from p75-mutant mice, but not absent. In contrast, over-expression of a truncated form of TROY lacking its intracellular domain (DN-TROY) could fully block this activation, unlike its full-length control.

Our data indicate that myelin inhibitors can trigger the phosphorylation of JNK via the NgR complex with either p75 or TROY.

### EXAMPLE 2

### Partial Inhibition of JNK Activation Promotes Axonal Regeneration After Spinal Injury in Rats

This animal study demonstrates that in an animal model for spinal injury, axonal regeneration can be promoted by intrathecal or intravenous administration of SP600125. Methodology for this animal study was adapted from Nash et al (J. Neurosci (2002) 22:7111-7120), Luo et al (Molecular Pain (2005) 1:29), and Obata et al (J. Neurosci. (2004) 24:10211-22).

Adult Sprague Dawley rats (300-400 gm) are trained and tested in a directed forepaw reaching (DFR) apparatus which measures grasping ability. The apparatus, which is described in detail by Nash et al., supra, is a box that consists of two compartments: a main compartment for housing the rats and a minor compartment for the food, separated by a Plexiglas divider. The minor compartment is subdivided into slots of equal size, each holding a pellet of food. Between the slots and the Plexiglas there is a gap. The apparatus is configured such that in order to retrieve a food pellet from a slot, a rat must extend a forelimb through a hole in the Plexiglas divider, and grasp the pellet and lift it over the gap and out of the slot. If the rat merely rakes the food in the slot towards the hole in the Plexiglas, the food will drop from the slot into the gap and fall to the floor of the minor compartment. The floor of the minor compartment can be configured to allow a rat to retrieve food that drops or it can be lowered to prevent the rat from reaching dropped food. Prior to inducing spinal injury, the rats are food restricted, receiving ˜3 gm food/100 gm body weight per day, before and throughout training and testing. Weight is monitored to ensure that rats are reduced to no less than 80% of their original body weight at any time. All rats are given shaping periods for 2-3 d in the box to allow them to learn the task while they become familiar with the testing situation. Animals are trained twice per day for 5 d and then tested twice per day for 5 d, and presurgical DFR data is collected. During the testing period, rats are given 5 min to complete the task and are allowed to make as many attempts as they want during this time period. Rats are required to return to at least 95% of their original weight to ensure that they are healthy before undergoing surgery.

Rats are randomly assigned to control or experimental groups. Sham control rats undergo surgical procedure without lesioning, and with or without placement of a mini osmotic pump (Alzet type 2001; Durect, Cupertino, Calif.). Lesioned control rats receive no treatment, tail vein injection with vehicle only treatment, insertion of a mini osmotic pump only treatment, or insertion of a mini osmotic pump with vehicle only treatment. After anesthesia with isoflurane, the rats are placed on an operating board in such a way as to bend the cervical spinal cord for maximum exposure. A laminectomy is performed exposing the dorsum of the spinal cord between C2 and C4. The dorsal columns are identified bilaterally, and, in all rats except for those in the sham group, a suture needle is passed through the spinal cord, isolating the dorsal funiculus. The suture thread is gently lifted, and a pair of iridectomy scissors is used to bilaterally transect the dorsal funiculus, thereby transecting the dorsal corticospinal tract (CST). Visualization of the dorsal horns and the central gray commissure confirms accuracy of the lesion borders. A pledget of biodegradable Gelfoam soaked in a fluorescent retrograde tracer, Fluorogold (3% in 0.9% saline; Molecular Probes), is placed in the lesion site to identify the neurons whose axons are transected, confirming the lesion. Rats designated for SP600125 treatment or corresponding control treatment are implanted with mini osmotic pumps adjacent to the lesion site. The pumps in the SP600125 treatment group operate at a rate of 1 μl/hr for a period of 7 days and are filled with SP600125 at a concentration of 500 ng/μl. The overlying muscles and skin are sutured, and the rats are placed on a heating pad to maintain body temperature. Each rat receives a single dose of buprenorphine (0.1 mg/kg) immediately after surgery to alleviate pain.

One hour after the spinal cord is lesioned, the rats in the tail vein injection treatment group receive a bolus injection of 100 μg/kg SP600125 in a saline/DMSO vehicle. The treatment is repeated every 24 hours on days 1 through 7 post-lesion. Vehicle only control rats undergo the same treatment but are injected with an equal volume of saline/DMSO in a tail vein.

Rats are trained twice per week during weeks 2-5 after surgery. Some rats may be profoundly impaired such that they may not be able to grasp food in the DFR task in the early postsurgical period. In this case, the apparatus can be configured to allow the rats to rake food into the main compartment that drops from the slot onto the floor of the minor compartment (see Nash et al., supra). This ensures that the reaching portion of the DFR task does not extinguish. The severity of the grasping impairment decreases as the postsurgical period increases, and the configuration of the apparatus that does not permit food raking can be gradually reestablished. By the end of the postsurgical recovery period, all rats are able to successfully perform the DFR task, to some degree. During the sixth week after surgery, rats are tested twice per day for 5 d, and postsurgical DFR data is collected by a blinded investigator. Just as during the presurgery testing period, the rats are allowed 5 min to complete the task during the postsurgery testing period and are allowed to make as many attempts as they want during this time period. The data is collected in terms of total number of attempts and percentage of successful attempts. An attempt is scored only when a rat reaches into a slot and displaces the pellet or drops it to the floor of the minor compartment. A successful attempt is scored when a rat grasps a pellet, lifts it over the gap and pulls it through the Plexiglas divider into the main portion of the testing apparatus.

Sham animals perform the DFR task as well postsurgically as they do presurgically, demonstrating that only the lesion, and no other portion of the surgical procedure, inhibits the rats' abilities to perform the DFR task. The lesion and vehicle groups are the most impaired of all of the groups after surgery. The lesion and vehicle groups are able to perform the DFR task with a success rate of only about 40%. Significantly better performance by the SP600125-treated group demonstrates the effect of the treatment on functional recovery after spinal injury.

Seven weeks after injury, rats are prepared for injection of biotin dextran tetramethylrhodamine (BDT; Molecular Probes). This fluorescent anterograde tracer, injected into the primary motor cortex, is used to label CST axons caudal to the lesion site in the spinal cord. After anesthesia with isoflurane (5%), rats are placed in a stereotaxic instrument, and a total of six stereotaxically determined holes (0.9 mm diameter) are drilled in the skull over the primary motor cortices associated with the forelimbs. The anteroposterior (AP) and mediolateral (ML) coordinates for these injections, from bregma, are as follows: ±0.5 AP and ±3.5 ML; ±1.5 A/P and ±2.5 ML; and ±2.5 AP and ±1.5 ML. All injections are delivered at a depth of 2.5 mm from the surface of the skull. A 10 μl Hamilton syringe is used to inject BDT bilaterally into layer V of the cortex. Three injections into each cortical hemisphere are used to administer a total of 1.2 μl of the anterograde tracer. Bone wax (Ethicon, Somerville, N.J.) is used to seal the holes in the skull, the scalp is sutured, and a single dose of buprenorphine (0.1 mg/kg) is administered immediately after surgery to alleviate pain. Rats are killed 3 d after tracer injections.

Seven weeks and 3 d after lesioning, rats are anesthetized with chloral hydrate (10 ml/kg) and perfused transcardially with 300 ml of PBS, pH 7.4, followed by 300 ml of 4% paraformaldehyde in 0.1 M phosphate buffer. After the animals are killed, all brains and spinal cords are removed and soaked overnight in 30% sucrose in a 0.1M phosphate buffer solution. The brains are cut coronally and the spinal cords are cut horizontally at a thickness of 20 μm with a freezing microtome and mounted on ProbeOn (Fisher Scientific, Pittsburgh, Pa.) coated slides. Brain and spinal cord sections are examined using a Nikon (Tokyo, Japan) Labophot fluorescent microscope, and images are captured using a digital still camera. The forelimb representation of the primary motor cortex is identified based on the stereotaxic BDT injection sites. The primary motor cortex is examined in all rats. Presence of Fluorogold-labeled neurons in layer V of the primary motor cortex, confirms that the dorsal CST axons were transected during the lesioning procedure. Because all CST axons located in the dorsal funiculus are transected during surgery and not just those in the forelimb representation, Fluorogold-labeled neurons are found throughout the primary motor cortex in layer V. The only exception to this labeling pattern is in the brains of the rats in the sham group whose brains have no Fluorogold label.

The spinal cord caudal to the lesion is examined, and the BDT-labeled axons occupying the region of the spinal cord normally occupied by the dorsal CST are counted. For each section, the number of BDT-labeled axons is counted at 3 mm intervals caudal to the lesion, beginning 1 mm distal to the injury (i.e., 1 mm, 4 mm, 7 mm, etc.) and ending 19 mm caudal to the lesion site. Innervation of the rat forepaw extends to T1, a distance of 15.1 mm from the lesion at C3. Therefore, analysis of the axons out to 19 mm caudal to the lesion ensures that the entire distance representing the forepaw is examined. At each interval, the total number of BDT-labeled axons (left and right CST combined) along a 500 μm length (length of microscope field) is counted. In each field counted, the focal plane is adjusted up and down to ensure that a single continuous axon is not double counted if it traverses out of the focal plane and reemerges farther down in the same field. The number of BDT-labeled axons present is examined for control and experimental groups at each of the distances (i.e. 1, 4, 7 . . . and 19 mm caudal to the lesion site). Throughout all of the examined intervals, the mean number of axons is highest in the sham group, and, at each distance examined, the mean number of labeled axons in the sham group is significantly higher than in the other groups. No significant difference is observed between the means of the lesion and vehicle groups at any distance examined. In these groups, axons are found only a short distance caudal to the injury, and, by 10 mm distal to the lesion to the farthest distance examined, all of the tissue is virtually devoid of axons. Significantly more labeled axons at each distance in the SP600125-treated group compared to lesioned control rats demonstrates that this treatment promotes axonal regeneration after spinal injury.

### EXAMPLE 3

### Improved Neurological Outcome Following SP600125 Treatment for Acute Spinal Cord Injury

We adapted our protocol for this study from the Sygen® Multicenter Acute Spinal Cord Injury Study described by Geisler et al (Spine (2001) 26:587-598). It is a prospective, double-blind, randomized, and stratified multicenter trial, randomizing approximately 800 patients so as to have at least 720 completed and evaluable in each of three initial treatment groups: placebo, low-dose SP600125, and high-dose SP600125. The patients are stratified into six groups, according to three degrees of injury severity (American Spinal Injury Association grades A, B, and C+D) and two levels of anatomic injury (cervical and thoracic). The trial is sequential with preplanned interim analyses as each group of 720/4=180 patients reach their 26-week examination and become evaluable. Patients are required to have at least one lower extremity with a substantial motor deficit. Patients with spinal cord transection or penetration are excluded, as are patients with a significant cauda equina, brachial or lumbosacral plexus, or peripheral nerve injury. Gunshot injuries that do not penetrate the cord are allowed. Multiple trauma is allowed as long as it is not so severe as to prevent neurologic measurement evaluation or interpretation.

All patients are to receive the second National Acute Spinal Cord Injury Studies (NASCIS II) dose regimen of methylprednisolone (MPSS) starting within 8 hours after the spinal cord injury (SCI). To avoid any possible untoward interaction between MPSS and SP600125 the study medication is not started until after completion of MPSS administration.

The placebo group has a loading dose of placebo and then 56 days of placebo. The low dose SP600125 group has a 50-mg loading dose administered intravenously (i.v.) followed by 10 mg/day i.v. for 56 days. The high dose SP600125 group has a 250-mg loading dose followed by 50 mg/day for 56 days.

The baseline neurologic assessment includes both the AIS and detailed American Spinal Injury Association (ASIA) motor and sensory examinations. Modified Benzel Classification and the ASIA motor and sensory examinations are performed at 4, 8, 16, 26, and 52 weeks after injury. The Modified Benzel Classification is used for post-baseline measurement because it rates walking ability and, in effect, subdivides the broad D category of the AIS. Because most patients have an unstable spinal fracture at baseline, it is not possible to assess walking ability at that time; hence the use of different baseline and follow-up scales. Marked recovery is defined as at least a two-grade equivalent improvement in the Modified Benzel Classification from the baseline AIS. The primary efficacy assessment is the proportion of patients with marked recovery at week 26. The secondary efficacy assessments include the time course of marked recovery and other established measures of spinal cord function (the ASIA motor and sensory scores, relative and absolute sensory levels of impairment, and assessments of bladder and bowel function).

### EXAMPLE 4

### Effect of JNK Inhibition After Cortical Impact Injury in Rats

We adapted methodology from Cherian et al. (J Pharmacol Exp Ther. (2003) 304:617-23), to test the effects of different doses and treatment schedules of AS601245 on a rat model of brain impact injury. A total of 60 male Evans rats weighing 300 to 400 g are assigned to one of the following doses injected intraperitoneally (i.p.) or intracerebral ventricularly (i.c.v.): none (saline control group), 0.01, 0.1, 1.0, and 10.0 mg/kg/day AS601245. The rats are further assigned to a treatment duration of 1, 3, 7, or 14 days, with 4 rats in each treatment group, and 3 rats in each control group (i.e. saline administered for 1, 3, 7, or 14 days).

The details of the methods to produce the impact injury have been previously described (Cherian et al., J. Neurotrauma (1996) 13:371-383). Briefly, the head of the rat is fixed in a stereotaxic frame by ear bars and incisor bar. A 10-mm diameter craniotomy is performed on the right side of the skull over the parietal cortex. An impactor tip having a diameter of 8 mm is centered in the craniotomy site perpendicular to the exposed surface of the brain at an angle of approximately 45 degrees to the vertical. The tip is lowered until it just touches the dural surface. The impactor rod is then retracted, and the tip advanced an additional 3 mm to produce a brain deformation of 3 mm during the impact. Gas pressure applied to the impactor is adjusted to 150 psi, giving an impact velocity of approximately 5 m/s and duration of approximately 150 to 160 ms.

Rats are fasted overnight and anesthetized with 3.5% isoflurane in 100% oxygen in a vented anesthesia chamber. Following endotracheal intubation with a 16-gauge Teflon catheter, the rats are mechanically ventilated with 2% isoflurane in 100% oxygen for the surgical preparation and for the impact injury. Intracranial pressure (ICP) is monitored by a 3F microsensor transducer (Codman & Schurtleff, Randolph, Mass.) inserted in the left frontal lobe, well away from the impact site. ICP is monitored during the impact injury as a measure of the severity of the injury. Rectal temperature is maintained at 36.5-37.5° C. by a heating pad, which is controlled by rectal thermistor. Brain temperature is kept constant at 37° C. with the help of a heating lamp directed at the head.

The rats that are to receive i.c.v. administration of AS601245 receive mini-pump implants using procedures described by Kitamura et al (J Pharmacol Sci (2006) 100:142-148). Briefly, the rats are fixed in a stereotaxic frame (David Kopf Instruments, Tujunja, Calif.). Guide cannulae are implanted into the left lateral ventricle (Bregma −0.8 mm, lateral 1.5 with a depth of 3.7 mm below the dura). Each cannula is then connected by a catheter to an ALZET® mini-osmotic pump implanted subcutaneously in the scapular region and configured to continuously infuse the drug to achieve the specified daily dose of AS601245 (or vehicle only for control groups).

For rats in the i.p. treatment group, each dose of AS601245 is dissolved in 1 ml of sterile 0.9% saline so that the volume delivered is the same for each group and only the dosage of AS601245 varies. The first dose is administered within 1 hour following impact injury. And once daily thereafter for the assigned treatment duration.

After removing all catheters and suturing the surgical wounds, the rats are allowed to awaken from anesthesia. For the first 3 days post injury, the rats are treated with butorphanol tartrate, 0.05 mg of i.m. every 12 h (twice a day), for analgesia and enrofloxacin 2.27%, 0.1 ml of IM qd, to reduce the risk of postoperative infections.

The outcome measures are performed by investigators who are blinded to the treatment group. At 2 weeks after the impact, the animals are deeply anesthetized with a combination of ketamine/xylazine/acepromazine and perfused transcardially with 0.9% saline, followed by 10% phosphate buffered formaldehyde. The entire brain is removed and fixed in 4% formalin. The fixed brains are examined grossly for the presence of contusion, hematoma, and herniation. The brains are photographed, sectioned at 2-mm intervals, and then embedded in paraffin. Hematoxylin and eosin (H&E) stained 9-μm thick sections are prepared for histologic examination. Particular care is made to include the largest cross-sectional area of cortical injury on the cut surface of the embedded sections. The H&E-stained coronal sections are digitized using a Polaroid Sprint Scanner (Polaroid Corporation, Waltham, Mass.) equipped with a PathScan Enabler (Meyer Instruments, Houston, Tex.). The injury volume is measured by determining the cross-sectional area of injury in each H&E-stained coronal image and multiplying by the thickness of the tissue between the slices. This slab volume technique is implemented on the image processing program Optimas 5.2 (Optimas Corporation, Seattle, Wash.). Neurons in the middle 1-mm segments of the CA1 and CA3 regions of the hippocampus are counted at a magnification of 200×. Neurons are identified by nuclear and cytoplasmic morphology, and individual cells are counted whether normal or damaged. Neurons with cytoplasmic shrinkage, basophilia, or eosinophilia or with loss of nuclear detail are regarded as damaged. The regions measured are 1 mm long and 1 mm wide (0.5 mm on either side of the long axis of the segment). The total number of neurons and the number of neurons that appear normal are expressed as neurons per squared millimeter.

### EXAMPLE 5

### JNK Inhibition Promotes Regeneration of Lesioned Optic Nerve Fibers in Adult Mice

We designed this study to demonstrate that inhibiting neuronal JNK activity can promote axon regeneration in a previously described model of optic nerve crushing [Fischer et al, J. Neurosci. 18, 1646 (2004)]. All animal experiments are done in accordance with protocols approved by the institutional animal care and use committee at Schepens Eye Research Institute. Adult mouse optic nerves are exposed behind the eyeball and crushed. Immediately after injury in adult mice, Gelfoam soaked in a solution containing 500 ng/ml SP600125 or 0.1% DMSO (control) is placed against the crush site of the nerve and replaced every three days for the first six days of the study. Animals are sacrificed two weeks post injury followed by transcardial perfusion with 4% paraformaldehyde. Optic nerves are cryosectioned at 10 mm and stained with an anti-GAP43 antibody (Chemicon) to detect regenerating axons [Fischer et al, supra]. Little regeneration is detected in DMSO-treated control mice. However, suppression of JNK activity by injury site application of SP600125 results in significant increases in axonal regrowth and the number of regenerating axons, measured 0.25 mm beyond the injury site, compared to control mice. To test the possibility that axon regrowth of retinal ganglion neurons (RGCs) after SP600125 treatment is a consequence of improved cell survival, we stain retinal sections with the anti-tubulin Tuj1 antibody, which stains RGCs in the retina, and count surviving RGCs. No detectable effect of SP600125 treatment on RGC survival is found, indicating that blocking JNK activity locally and within a short time window following injury is sufficient to promote significant regeneration of lesioned optic nerve fibers in adult mice.

### EXAMPLE 6

### JNK Inhibition Promotes Neural Regeneration in Animal Models of Focal Brain Ischemia

This study uses previously described methods (Brines et al, Proc Natl Acad Sci USA. (2000) 97:10526-31) to demonstrate the effect of a systemically administered JNK inhibitor in an animal model of focal brain ischemia.

Sprague-Dawley male rats weighing ˜250 g are anesthetized with pentobarbital [60 mg/kg body weight (BW)]. Body core temperature is thermostatically maintained at 37° C. by using a water blanket and a rectal thermistor (Harvard Apparatus) for the duration of the anesthesia. The carotid arteries are visualized, and the right carotid is occluded by two sutures and cut. A burr hole adjacent and rostral to the right orbit allows visualization of the MCA, which is cauterized distal to the rhinal artery. Animals are then positioned on a stereotaxic frame. To produce a penumbra surrounding this fixed MCA lesion, the contralateral carotid artery is occluded for 1 h by using traction provided by a fine forceps. 0.5 ml of a 1 μg/ml solution of SP600125 or vehicle control is administered at 1 hr, 1 day, 5 days, or 10 days from the onset of the reversible carotid occlusion. To evaluate the extent of injury, the animals are killed after 15 days, the brains are removed, and serial 1-mm thick sections through the entire brain are cut by using a brain matrix device (Harvard Apparatus). Each section is then incubated in a solution of 2% triphenyltetrazolium chloride (wt/vol) in 154 mM NaCl for 30 min at 37° C. and stored in 4% paraformaldehyde until analysis. Quantification of the extent of injury is determined by using a computerized image analysis system (MCID, Imaging Research, St. Catharine's, ON, Canada). To accomplish this, a digital image of each section is obtained and the area of injury delineated by outlining the region in which the tetrazolium salt is not reduced, i.e., nonviable tissue. For cases in which the necrosis is so severe that tissue is actually lost and therefore the borders can not be directly assessed, an outline of the contralateral side is used to estimate the volume of injured brain. Total volume of infarct is calculated by reconstruction of the serial 1-mm thick sections.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims

### REFERENCES

1. Filbin, M. T. Nat Rev Neurosci 4, 703-13 (2003).
2. He, Z. & Koprivica, V. Annu Rev Neurosci 27, 341-68 (2004).
3. Yiu, G. & He, Z. S. Curr Opin Neurobiol 13, 545-51 (2003).
4. Domeniconi, M. et al. Neuron 35, 283-90 (2002).
5. Liu, B. P. et al. Science 297, 1190-3 (2002).
6. Wang, K. C. et al. Nature 417, 941-4 (2002).
7. Fournier, A. E. et al. Nature 409, 341-6 (2001).
8. Dergham, P. et al. J Neurosci 22, 6570-7 (2002).
9. Fournier, A. E. et al. J Neurosci 23, 1416-23 (2003).
10.Lehmann, M. et al. J Neurosci 19, 7537-47 (1999).
11.Niederost, B. et al. J Neurosci 22, 10368-76 (2002).
12.Winton, M. J. et al. J Biol Chem 277, 32820-9 (2002).
13. Hall, A. Science 279, 509-14 (1998).
14. Hsieh, S. H. et al. J Neurosci 26, 1006-15 (2006).
15. Wang, K. C. et al. Nature 420, 74-8 (2002).
16. Wong, S. T. et al. Nat Neurosci 5, 1302-8 (2002).
17. Yamashita, T. et al. J Cell Biol 157, 565-70 (2002).
18. Park, J. B. et al. Neuron 45, 345-51 (2005).
19. Shao, Z. et al. Neuron 45, 353-9 (2005).
20. Yamashita, T. & Tohyama, M. Nat Neurosci 6, 461-7 (2003).
21. Dempsey, P. W. et al. Cytokine Growth Factor Rev 14, 193-209 (2003).
22. Locksley, R. M. et al. Cell 104, 487-501 (2001).
23. Morrison, D. K. & Davis, R. J. Annu Rev Cell Dev Biol 19, 91-118 (2003).
24. Bennett, B. L. et al. Proc Natl Acad Sci USA 98, 13681-6 (2001).
25. Han, Z. et al. J Clin Invest 108, 73-81 (2001)
26. Chang, L. et al. Dev Cell 4, 521-33 (2003).
27. Yoshida, H. et al. J Neurochem 90, 352-8 (2004).
28. Gdalyahu, A. et al. Embo J 23, 823-32 (2004).
29. Kallunki, T. et al. Cell 87, 929-39 (1996).
30. Verhey, K. J. et al. Trends Biochem Sci 26, 545-50 (2001).
31. Goldberg, J. L. et al. Genes Dev 17, 941-58 (2003).
32. Schwaiger, F. W. et al. Prog Brain Res 117, 197-210 (1998).
33. Stoll, G. & Muller, H. W. Brain Pathol 9, 313-25 (1999).
34. Raivich, G. et al. Neuron 43, 57-67 (2004).
35. Lindwall, C. & Kanje, M. Mol Cell Neurosci 29, 269-82 (2005).
36. Herdegen, T. et al. Trends Neurosci 20, 227-31 (1997).

What is claimed is:

1. A method of promoting regeneration of a lesioned CNS axon of a mature neuron determined to be subject to regeneration inhibition by endogenous cJun-N-terminal kinase (JNK), the method comprising the steps of:
(a) contacting the neuron with an exogenous JNK signaling pathway inhibitor at a concentration sufficient to only partially inhibit the JNK signaling, and thereby promote a resultant regeneration of the axon; and
(b) detecting the resultant regeneration of the axon.

2. The method of claim 1 wherein the inhibitor is selected from the group consisting of CEP-1347, SP600125, AS601245, and DJNK1 peptide.

3. The method of claim 1 wherein the inhibitor is SP600125 at a nanomolar concentration.

4. The method of claim 1 wherein the lesion results from a traumatic injury.

5. The method of claim 1 wherein the lesion results from a traumatic brain injury.

6. The method of claim 1 wherein the lesion results from a stroke.

7. The method of claim 1 wherein the lesioned axon is in the optic nerve.

8. The method of claim 1 wherein the lesion results from an acute spinal cord injury.

9. The method of claim 1 wherein the lesioned axon is in the spinal cord of a patient, and the inhibitor is intrathecally administered to the patient.

10. The method of claim 1 wherein the axon is a CNS axon of a sensory neuron.

11. The method of claim 1 wherein the lesion results from CNS degeneration.

12. The method of claim 1 wherein the inhibitor is administered intravenously to a patient.

13. The method of claim 1 wherein the inhibitor is administered intrathecally to a patient.

14. The method of claim 1 wherein the inhibitor is administered ocularly to a patient.

15. The method of claim 1 wherein the detecting step is effected by an indirect assay of axon regeneration.

16. The method of claim 1 wherein the detecting step is effected by a direct assay of axon regeneration.
